(19) United States
(12) Patent Application Publication
(10) Pub. No.: US 2008/0199873 A1
(43) Pub. Date: Aug. 21, 2008

(54) Companion diagnostic assays for cancer therapy

(76) Inventors: Mark G. Anderson, Grayslake, IL (US); Paul E. Kroeger, Libertyville, IL (US); Saul Howard Rosenberg, Grayslake, IL (US); Stephen K. Tahir, Waukegan, IL (US); Christin Tse, Libertyville, IL (US); John A. Wass, Lake Forest, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/999,330

(22) Filed: Dec. 4, 2007

Related U.S. Application Data
(60) Provisional application No. 60/872,668, filed on Dec. 4, 2006.

(51) Int. Cl. C12Q 1/68; G06F 19/00

(52) U.S. Cl. 435/6; 702/20

(57) ABSTRACT

Methods for identifying cancer patients eligible to receive Bcl-2 family inhibitor therapy and for monitoring patient response to Bcl-2 family inhibitor therapy comprise assessment of the expression levels of the biomarker combinations set out in TABLES 1, 2, 3, 4, 5 or 6 in a patient tissue sample. The methods of the invention allow more effective identification of patients to receive Bcl-2 family inhibitor therapy and of determination of patient response to the therapy.

### FIELD OF THE INVENTION

This invention relates to diagnostic assays useful in classification of patients for selection of cancer therapy, and in particular relates to measurements of expression signatures, particularly biomarker combinations, where the signatures correlate with responsiveness to cancer therapy and particularly Bcl-2-family antagonist therapy. Additionally, methods of the present invention, and particularly the biomarker combinations, are useful in the identification of patients eligible to receive Bcl-2-family antagonist therapy and that permit monitoring of patient response to such therapy.

### BACKGROUND OF THE INVENTION

Genetic heterogeneity of cancer is a factor complicating the development of efficacious cancer drugs. Cancers that are considered to be a single disease entity according to classical histopathological classification often reveal multiple genomic subtypes when subjected to molecular profiling. In some cases, molecular classification proved to be more accurate than the classical pathology. The efficacy of targeted cancer drugs may correlate with the presence of a genomic feature, such as a gene amplification, Cobleigh, M. A., et al., “Multinational study of the efficacy and safety of humanized anti-HER2 monoclonal antibody in women who have HER2-overexpressing metastatic breast cancer that has progressed after chemotherapy for metastatic disease”, J. Clin. Oncol., 17: 2639-2648, 1999; or a mutation, Lynch, T. J., et al., “Activating mutations in the epidermal growth factor receptor underlying responsiveness of non-small-cell lung cancer to gefitinib”, N. Engl. J. Med., 350: 2129-2139, 2004. For Her-2 in breast cancer, it has been demonstrated that detection of gene amplification provides superior prognostic and treatment selection information as compared with the detection by immunohistochemistry (IHC) of the protein overexpression, Pauletti, G., et al., “Assessment of Methods for Tissue-Based Detection of the HER-2/neu Alteration in Human Breast Cancer: A Direct Comparison of Fluorescence In Situ Hybridization and Immunohistochemistry”, J. Clin. Oncol., 18: 3651-3664, 2000. Cell line expression pattern data has specifically been shown to be predictive for patient sensitivity to chemotherapeutics Potti A., et al., Nat. Med. 2006 Epub ahead of print, PMID: 17057710. A need therefore exists for genomic classification markers that may improve the response rate of patients to targeted cancer therapy.

Targeted cancer therapy research has been reported against members of the Bcl-2 protein family, which are central regulators of programmed cell death. The Bcl-2 family members that inhibit apoptosis are overexpressed in cancers and contribute to tumorigenesis. Bcl-2 expression has been strongly correlated with resistance to cancer therapy and decreased survival.

A compound called ABT-737 is a small-molecule inhibitor of the Bcl-2 family members Bcl-2, Bcl-XL, and Bcl-w, and has been shown to induce regression of solid tumors, Oltersdorf, T., “An inhibitor of Bcl-2 family proteins induces regression of solid tumors”, Nature, 435: 677-681, 2005. ABT-737 has been tested against a diverse panel of human cancer cell lines and has displayed selective potency against SCLC and lymphoma cell lines, Ibid. ABT-737's chemical structure is provided by Oltersdorf et al. at p. 679.

Because of the potential therapeutic use of inhibitors for Bcl-2 family members, companion diagnostic assays that would identify patients eligible to receive Bcl-2 family inhibitor therapy are needed. Additionally, there is a clear need to support this therapy with diagnostic assays using biomarkers that would facilitate monitoring the efficacy of Bcl-2 family inhibition therapy.

### SUMMARY OF THE INVENTION

The present invention relates to the identification and use of gene expression patterns (or profiles or signatures), which are clinically relevant to cancer therapy. In particular, the identities of genes that are correlated with the identification, treatment and monitoring of patients for cancer treatment and particularly Bcl-2 family antagonist therapy.

The invention provides companion diagnostic assays for classification of patients for cancer treatment which comprise assessment in a patient tissue sample the levels of biomarker combinations set out in TABLES 1, 2, 3, 4, 5 or 6. The inventive assays include assay methods for identifying patients eligible to receive Bcl-2 family antagonist therapy and for monitoring patient response to such therapy. The invention methods comprise assessment of the biomarkers in blood, urine, or other body fluid samples by immunoassay, proteomic assay or nucleic acid hybridization or amplification assays, and in tissue or other cellular body samples by immunohistochemistry or in situ hybridization assays.

Gene expression patterns of the invention are identified as described below. Generally a large sampling of the gene expression profile of a sample is obtained through quantifying the expression levels of mRNA corresponding to many genes identified in the biomarker combinations. The profile, or combination set is then analyzed to identify genes, the expression of which are positively correlated with the identification and monitoring of patients eligible of cancer treatment and particularly Bcl-2 family antagonist therapy.

In a preferred embodiment, the invention comprises a method for identifying a patient as eligible to receive cancer therapy, and preferably Bcl-2 family inhibitor therapy comprising: (a) providing a peripheral blood sample from a patient; (b) determining expression levels in the peripheral blood sample of biomarker combinations set out in TABLES 1, 2, 3, 4, 5 or 6; and (c) classifying the expression levels relative to normal peripheral blood levels of biomarker combinations set out in TABLES 1, 2, 3, 4, 5 or 6; and (d) identifying the patient as eligible for cancer therapy and preferably Bcl-2 family inhibitor therapy where the patient's blood sample is classified as having elevated expression levels of biomarker combinations set out in TABLES 1, 2, 3, 4, 5 or 6. In this embodiment, levels in the peripheral blood sample is preferably determined by a polymerase chain reaction (PCR) assay, for example, or performed on a lung cancer tumor biopsy sample.

In a preferred embodiment, the invention comprises a method for identifying a patient as eligible for cancer therapy and most preferably Bcl-2 family inhibitor therapy comprising: (a) providing a tissue or cellular sample from a patient; (b) contacting the tissue or cellular sample with a labeled antibody or protein capable of binding to the biomarker combinations set out in TABLES 1, 2, 3, 4, 5 or 6; (c) classifying the expression levels relative to normal tissue or cellular level of the biomarker combinations set out in TABLES 1, 2, 3, 4, 5 or 6; and (d) identifying the patient as eligible for cancer therapy and most preferably Bcl-2 family therapy where the patient's sample is classified as having differential levels of members of the biomarker combinations set out in TABLES 1, 2, 3, 4, 5 or 6.

The invention has significant capability to provide improved stratification of patients for cancer therapy, and in particular for Bcl-2 family inhibitor therapy. The assessment of these biomarkers with the invention also allows tracking of individual patient response to the therapy. The inventive assays have particular utility for classification of small cell lung carcinoma (SCLC) and leukemia/lymphoma patients.

The invention also comprises a preferred method for monitoring a patient being treated for cancer and preferably with Bcl-2 family inhibitor therapy comprising: (a) providing a peripheral blood sample from a patient; (b) measuring expression levels in the peripheral blood sample of the biomarker combinations set out in TABLES 1, 2, 3, 4, 5 or 6; and (c) determining the expression level relative to a patient baseline blood level of the biomarker combinations set out in TABLES 1, 2, 3, 4, 5 or 6.

The invention also comprises a reagent kit for an assay for levels of the RNA from the biomarker combinations set out in TABLES 1, 2, 3, 4, 5 or 6, as well as a reagent kit for levels if at least one RNA from the biomarker combinations set out in TABELS 1, 2, 3, 4, 5 or 6. The invention has significant capability to provide improved stratification of patients for cancer therapy, and in particular for Bcl-2 family inhibitor therapy. The assessment of these biomarkers with the invention also allows tracking of individual patient response to the therapy. The inventive assays have particular utility for classification of SCLC and lymphoma patients.

### BRIEF DESCRIPTION OF THE DRAWINGS

FIG. 1 shows the expression profile for the biomarker combination groups that differentiate line sensitive and resistant to ABT-737 for small cell lung carcinoma cells (FIG. 1-A) and leukemia/lymphoma cells (FIG. 1-B).

FIG. 2 shows the expression profile for the biomarker combination groups that differentiate line sensitive and resistant to ABT-263 for small cell lung carcinoma cells (FIG. 2-A) and leukemia/lymphoma cells (FIG. 2-B).

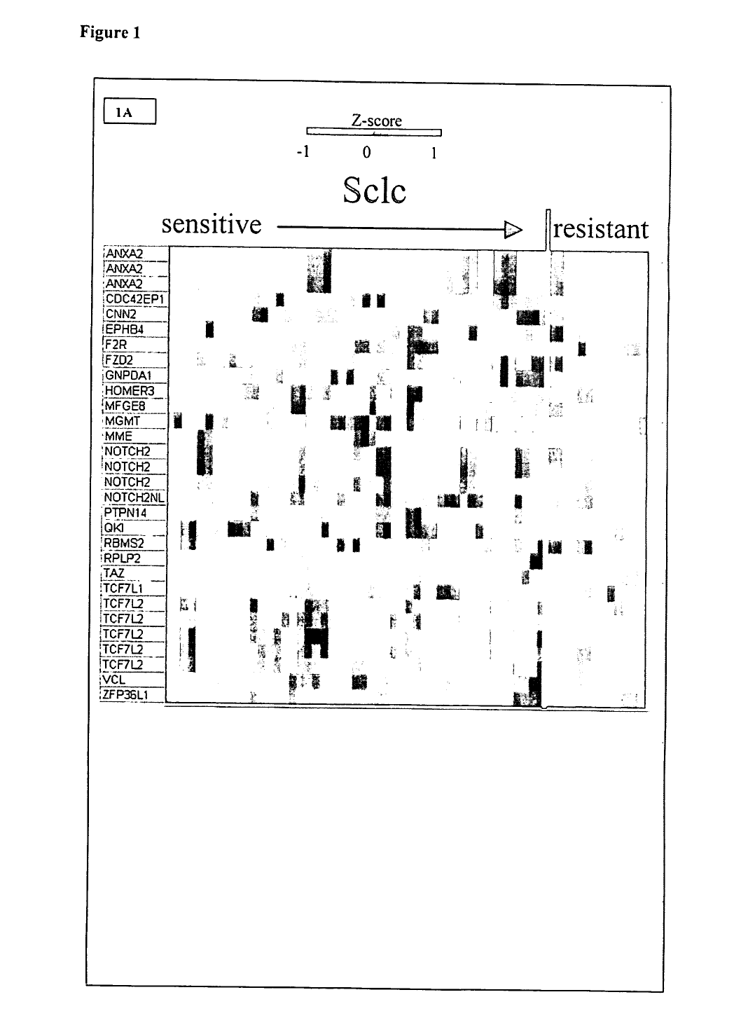

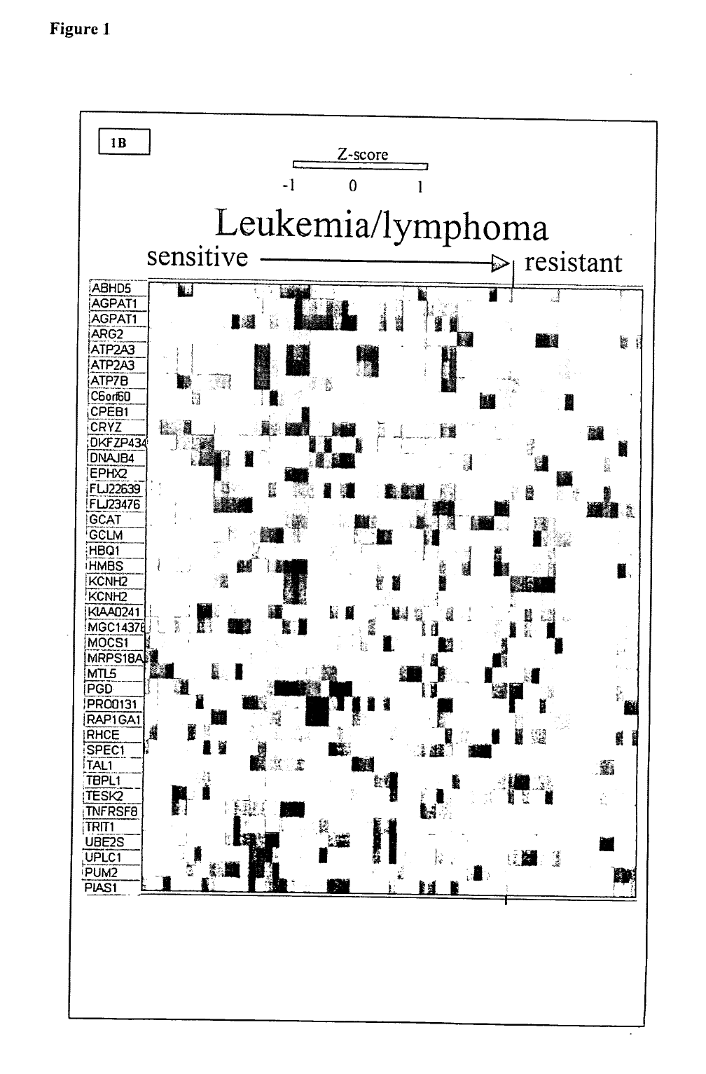

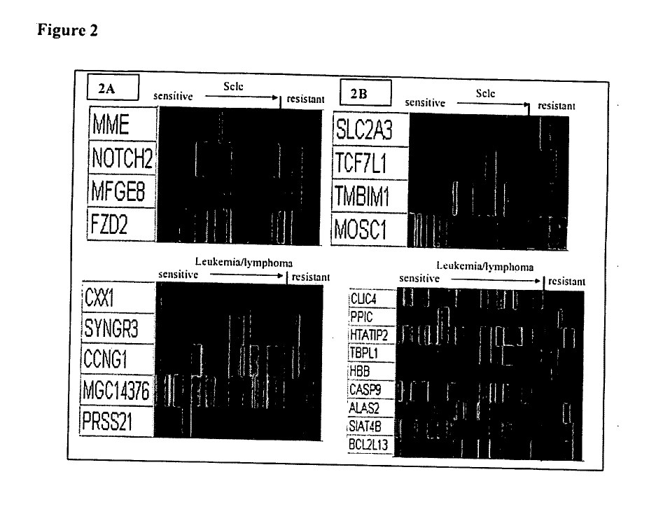

### DETAILED DESCRIPTION OF THE INVENTION

I. General

The invention is based on the discovery by Applicants of gene and gene signature groups in small cell lung cancer cell (sclc) lines and leukemia/lymphoma cell lines that correlate to therapy resistance and sensitivity. In particular, Applicants correlated differential expression levels of novel biomarker combinations, which correlate to the sensitivity and resistance to a Bcl-2 family inhibitor.

As used herein, a “Bcl-2 family inhibitor” refers to a therapeutic compound of any type, including small molecule-, antibody-, antisense-, small interfering RNA-, or microRNA-based compounds, that binds to at least one of Bcl-2, Bcl-XL, and Bcl-w, and antagonizes the activity of the Bcl-2 family related nucleic acid or protein. The inventive methods are useful with any known or hereafter developed Bcl-2 family inhibitor. One Bcl-2 family inhibitor is ABT-737, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide, which binds to each of Bcl-2, Bcl-XL, and Bcl-w. Another Bcl-2 family inhibitor is ABT-263, N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide. The chemical structure of ABT-263 is:

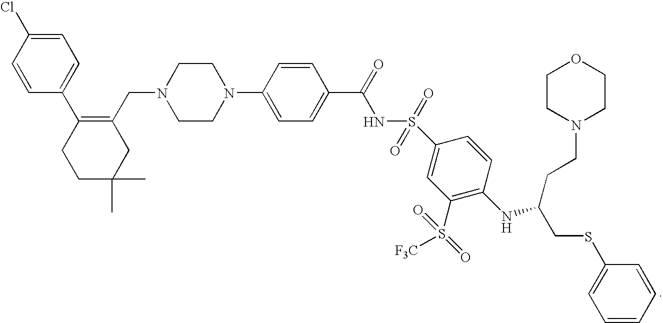

Other examples of Bcl-2 family related compounds useful in the present invention can be found in International Publication Numbers WO 05/049593 and WO 05/049594, both published on Jun. 2, 2005, incorporated by reference herein in its entirety.

The assays of the invention have potential use with targeted cancer therapy. In particular, the inventive assays are useful with therapy selection for small cell lung cancer and leukemia/lymphoma patients, such as therapy with Bcl-2 family inhibitors. Other examples of such cancers include solid tissue epithelial cancers, e.g., prostate, ovarian and esophageal cancer. The inventive assays are performed on a patient tissue sample of any type or on a derivative thereof, including peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates.

As used herein, Bcl-2 (official symbol BCL2) means the human B-cell CLL/lymphoma 2 gene; Bcl-x1 (official symbol BCL2L1) means the human BCL2-like 1 gene; Bcl-w (official symbol BCL2L2) means the human BCL2-like 2 gene.

As used herein, ANXA2 (official symbol ANXA2) annexin A2; CDC42EP1 (official symbol CDC42EP1); CDC42 (official symbol CDC42) effector protein (Rho GTPase binding 1); CNN2 (official symbol CNN2) calponin 2; EPHB4 (official symbol EPHB4) EPH receptor B4; F2R (official symbol F2R) coagulation factor II (thrombin) receptor; FZD2 (official symbol FZD2) frizzled homolog 2 (Drosophila); GNPDA1 (official symbol GNPDA1) glucosamine-6-phosphate deaminase 1; HOMER3 (official symbol HOMER3) homer homolog 3 (Drosophila); MFGE8 (official symbol MFGE8) milk fat globule-EGF factor 8 protein; MGMT (official symbol MGMT) O-6-methylguanine-DNA methyltransferase; MME (official symbol MME) membrane metallo-endopeptidase (neutral endopeptidase, enkephalinase, CALLA, C); NOTCH2 (official symbol NOTCH2) Notch homolog 2 (Drosophila); PTPN14 (official symbol PTPN14) protein tyrosine phosphatase, non-receptor type 14; QKI (official symbol QKI) quaking homolog, KH domain RNA binding (mouse); RBMS2 (official symbol RBMS2) RNA binding motif, single stranded interacting protein 2; TCF7L1 (official symbol TCF7L1) transcription factor 7-like 1 (T-cell specific, HMG-box); TCF7L2 (official symbol TCF7L2) transcription factor 7-like 2 (T-cell specific, HMG-box); VCL (official symbol VCL) vinculin; VIM (official symbol VIM) vimentin; WWTR1 (official symbol WWTR1) WW domain containing transcription regulator 1; ZFP36L1 (official symbol ZFP36L1) zinc finger protein 36, C3H type-like 1; PGD (official symbol PGD) phosphogluconate dehydrogenase; UBE2S (official symbol UBE2S) ubiquitin-conjugating enzyme E2S; CRYZ (official symbol CRYZ) crystallin, zeta (quinone reductase); HMBS (official symbol HMBS) hydroxymethylbilane synthase; DNAJB4 (official symbol DNAJB4) DnaJ (Hsp40) homolog, subfamily B, member 4; RAP1GA1 (official symbol RAP1GA1) RAP1, GTPase activating protein 1; GCLM (official symbol GCLM) glutamate-cysteine ligase, modifier subunit; ARG2 (official symbol ARG2) arginase, type II; ATP7B (official symbol ATP7B) ATPase, Cu++ transporting, beta polypeptide (Wilson disease); GCAT (official symbol GCAT) glycine C-acetyltransferase (2-amino-3-ketobutyrate coenzyme A ligase); KCNH2 (official symbol KCNH2) potassium voltage-gated channel, subfamily H (eag-related), member 2; TESK2 (official symbol TESK2) testis-specific kinase 2; TAL1 (official symbol TAL1) T-cell acute lymphocytic leukemia 1; TNFRSF8 (official symbol TNFRSF8) tumor necrosis factor receptor superfamily, member 8; ATP2A3 (official symbol ATP2A3) ATPase, Ca++ transporting, ubiquitous; TBPL1 (official symbol TBPL1) TBP-like 1; EPHX2 (official symbol EPHX2) epoxide hydrolase 2, cytoplasmic; KCNH2 (official symbol KCNH2) potassium voltage-gated channel, subfamily H (eag-related), member 2; MOCS1 (official symbol MOCS1) molybdenum cofactor synthesis 1; KIAA0241 (official symbol KIAA0241) KIAA0241 protein; MGC14376 (official symbol MGC14376) hypothetical protein MGC14376; YOD1 (official symbol YOD1) YOD1 OTU deubiquinating enzyme 1 homolog (yeast); AGPAT1 (official symbol AGPAT1) 1-acylglycerol-3-phosphate O-acyltransferase 1 (lysophosphatidic acid acyltransferase, alpha); RHCE (official symbol RHCE) Rhesus blood group, CcEe antigens; CDC42SE1 (official symbol CDC42SE1) CDC42 small effector 1; TRIT1 (official symbol TRIT1) tRNA isopentenyltransferase 1; YRDC (official symbol YRDC) ischemia/reperfusion inducible protein; ABHD5 (official symbol ABHD5) abhydrolase domain containing 5; DDEFL1 (official symbol DDEFL1) development and differentiation enhancing factor-like 1; CPEB 1 (official symbol CPEB1) cytoplasmic polyadenylation element binding protein 1; CCDC21 (official symbol CCDC21) coiled-coil domain containing 21; MTL5 (official symbol MTL5) metallothionein-like 5, testis-specific (tesmin); C6orf60 (official symbol C6orf60) chromosome 6 open reading frame 60; FLJ22639 (official symbol FLJ22639) hypothetical protein FLJ22639; HBQ1 (official symbol HBQ1) hemoglobin, theta 1; MRPS18A (official symbol MRPS18A) mitochondrial ribosomal protein S18A; AGPAT1 (official symbol AGPAT1) 1-acylglycerol-3-phosphate O-acyltransferase 1 (lysophosphatidic acid acyltransferase, alpha); PIAS1 (official symbol PIAS1) protein inhibitor of activated STAT, 1; PUM2 (official symbol PUM2) pumilio homolog 2 (Drosophila); SLC2A3 (official symbol SLC2A3) solute carrier family 2 (facilitated glucose transporter), member 3; transcription factor 7-like 2 (T-cell specific, HMG-box); TMBIM1 (official symbol TMBIM1) transmembrane BAX inhibitor motif containing 1; MOSC1 (official symbol MOSC1) MOCO sulphurase C-terminal domain containing 1; CXX1 (official symbol CXX1) CAAX box 1; SYNGR3 (official symbol SYNGR3) synaptogyrin 3; CCNG1 (official symbol CCNG1) cyclin G1; MGC14376 (official symbol MGC14376) hypothetical protein MGC14376; PRSS21 (official symbol PRSS21) protease, serine, 21 (testisin); CASP9 (official symbol CASP9) caspase 9, apoptosis-related cysteine peptidase; ALAS2 (official symbol ALAS2) aminolevulinate, delta-, synthase 2 (sideroblastic/hypochromic anemia); ST3GAL2 (official symbol ST3GAL2) ST3 beta-galactoside alpha-2,3-sialyltransferase 2; BCL2L13 (official symbol BCL2L13) BCL2-like 13 (apoptosis facilitator); PPIC (official symbol PPIC) peptidylprolyl isomerase C (cyclophilin C); CLIC4 (official symbol) chloride intracellular channel 4; TBPL1 (official symbol TBPL1) TBP-like 1; HBB (official symbol HBB) hemoglobin, beta///hemoglobin, beta; and HTATIP2 (official symbol HTATIP2) HIV-1 Tat interactive protein 2, 30 kDa.

As used herein, “consisting essentially of” refers to the maximum number of genes that are required for the use of a biomarker to improve stratification of patents for cancer therapy, and in particular Bcl-2 family inhibitor therapy. In one embodiment, a biomarker to improve stratification of patents for cancer therapy, and in particular Bcl-2 family inhibitor therapy consisting essentially of at least 1, 2, 3, 4, 5, 6, 7, or all of the biomarkers of the invention. In another embodiment, a biomarker to improve stratification of patients for cancer therapy, and in particular Bcl-2 family inhibitor therapy consisting essentially of any one of the biomarkers in TABLE 1. In another embodiment, a biomarker to improve stratification of patients for cancer therapy, and in particular Bcl-2 family inhibitor therapy consisting essentially of any one of the biomarkers in TABLE 2. In another embodiment, a biomarker to improve stratification of patients for cancer therapy, and in particular Bcl-2 family inhibitor therapy consisting essentially of any one of the biomarkers in TABLE 3. In another embodiment, a biomarker to improve stratification of patients for cancer therapy, and in particular Bcl-2 family inhibitor therapy consisting essentially of any one of the biomarkers in TABLE 4. In another embodiment, a biomarker to improve stratification of patients for cancer therapy, and in particular Bcl-2 family inhibitor therapy consisting essentially of any one of the biomarkers in TABLE 5. In another embodiment, a biomarker to improve stratification of patients for cancer therapy, and in particular Bcl-2 family inhibitor therapy consisting essentially of any one of the biomarkers in TABLE 6.

The biomarker combinations set out in Tables 1, 2, 3, 4, 5 or 6 may be used alone or in combination with each other.

As used herein, the term “differential expression” refers to a difference in the level of expression of the RNA of one or more biomarkers of the invention, as measured by the amount or level mRNA, and/or one or more spliced variants of mRNA of the biomarker in one sample as compared with the level of expression of the same one or more biomarkers of the invention in a second sample. “Differentially expressed” can also include a measurement of the protein encoded by the biomarker of the invention in a sample or population of samples as compared with the amount or level of protein expression in a second sample or population of samples. Differential expression can be determined as described herein and as would be understood by a person skilled in the art.

The term “gene” refers to a nucleic acid (e.g., DNA) sequence that comprises coding sequences necessary for the production of a polypeptide, RNA (e.g., including but not limited to, mRNA, tRNA and rRNA) or precursor (e.g., precursors). The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In particular, the term “gene” refers to the full-length nucleotide sequence. However, it is also intended that the term encompass fragments of the sequence, as well as other domains within the full-length nucleotide sequence. Furthermore, the terms “nucleotide sequence” or “polynucleotide sequence” encompasses DNA, cDNA, and RNA (e.g., mRNA) sequences.

As used herein, a “gene expression pattern” or “gene expression profile” or “gene signature” refers to the relative expression of genes correlated with the classification of patients for cancer therapy and particularly Bcl-2-family antagonist therapy, as well as the expression of genes correlation with the responsiveness and monitoring of patients undergoing cancer therapy and particularly Bcl-2-family inhibitor therapy. Moreover, the terms “gene expression pattern” or “gene expression profile” or “gene signature” indicate that combined pattern of the results of the analysis of the level of expression of two or more biomarkers of the invention including 3, 4, 5, 6, 7, 8, 9, 10, 11, 12 or all of the biomarkers of the invention. A gene expression pattern or gene expression profile or gene signature can result from the measurement of expression of the RNA and/or the protein expressed by the gene corresponding to the biomarkers of the invention. In the case of RNA it refers to the RNA transcripts transcribed from genes corresponding to the biomarker of the invention. In the case of protein it refers to proteins translated from the genes corresponding to the biomarker of the invention. For example, techniques to measure expression of the RNA products of the biomarkers of the invention includes, PCR based methods (including RT-PCR) and non PCR based methods as well as microarray analysis. To measure protein products of the biomarkers of the invention, techniques include western blotting and ELISA analysis.

Because the invention relies upon the identification of genes that are over expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other gene sequences. The term “about” as used in the preceding sentence refers to an increase or decrease of 10% from the stated numerical value. Longer polynucleotides may of course contain minor mismatches (e.g. via the presence of mutations), which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. Preferably, the sequences are those of mRNA encoded by the genes, the corresponding cDNA to such mRNAs, and/or amplified versions of such sequences. In preferred embodiments of the invention, the polynucleotide probes are immobilized on an array, other solid support devices, or in individual spots that localize the probes.

In another embodiment of the invention, all or part of a disclosed sequence may be amplified and detected by methods such as the polymerase chain reaction (PCR) and variations thereof, such as, but not limited to, quantitative PCR (Q-PCR), reverse transcription PCR (RT-PCR), and real-time PCR, optionally real-time RT-PCR. Such methods would utilize one or two primers that are complementary to portions of a disclosed sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization.

Alternatively, and in yet another embodiment of the invention, gene expression may be determined by analysis of expressed protein in a cell sample of interest by use of one or more antibodies specific for one or more epitopes of individual gene products (proteins) in said cell sample. Such antibodies are preferably labeled to permit their easy detection after binding to the gene product.

As used herein, the term “in combination” when referring to therapeutic treatments refers to the use of more than one type of therapy (e.g., more than one prophylactic agent and/or therapeutic agent). The use of the term “in combination” does not restrict the order in which therapies (e.g., prophylactic and/or therapeutic agents) are administered to a subject. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject.

Moreover, Bcl-2 inhibitor family therapy may also be administered in combination with one or more than one additional therapeutic agents, wherein additional therapeutic agents include radiation or chemotherapeutic agents, wherein chemotherapeutic agents include, but are not limited to, carboplatin, cisplatin, cyclophosphamide, dacarbazine, dexamethasone, docetaxel, doxorubicin, etoposide, fludarabine, irinotecan, CHOP (C: Cytoxan® (cyclophosphamide); H: Adiamycin® (hydroxydoxorubicin); O: Vincristine (Oncovin®); P: prednisone), paclitaxel, rapamycin, Rituxin® (rituximab) and vincristine.

As used herein, the term “level of expression” when referring to RNA refers to the measurable quantity of a given nucleic acid as determined by hybridization or measurements such as real-time RT PCR, which includes use of both SYBR.RTM. green and TaqMan.RTM. technology and which corresponds in direct proportion with the extent to which the gene is expressed. The level of expression of a nucleic acid is determined by methods well known in the art. For microarray analysis, the level of expression is measured by hybridization analysis using labeled nucleic acids corresponding to RNA isolated from one or more individuals according to methods well known in the art. The label on the nucleic acid used for hybridization can be a luminescent label, an enzymatic label, a radioactive label, a chemical label or a physical label. Preferably, target nucleic acids are labeled with a fluorescent molecule. Preferred fluorescent labels include, but are not limited to: fluorescein, amino coumarin acetic acid, tetramethylrhodamine isothiocyanate (TRITC), Texas Red, Cyanine 3 (Cy3) and Cyanine 5 (Cy5).

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

A “microarray” refers to an ordered arrangement of hybridizable array elements, preferably polynucleotide probes, on a support.

As used herein, the term “official symbol” refers to EntrezGene database maintained by the United States National Center for Biotechnology Information.

The term “support” refers to conventional supports such as beads, particles, dipsticks, fibers, filters, membranes and silane or silicate supports such as glass slides.

The invention comprises diagnostic assays performed on a patient tissue sample of any type or a derivate thereof, including peripheral blood, tumor or suspected tumor tissues (including fresh frozen and fixed or paraffin embedded tissue), cell isolates such as circulating epithelial cells separated or identified in a blood sample. Lymph node tissue, bone marrow and fine needle aspirates. Preferred tissue samples for use herein are peripheral blood, tumor or suspected tumor tissue and bone marrow.

II. Bcl-2 Family Inhibitor Biomarkers

Applicants identified novel biomarker combinations useful for stratifying and/or monitoring patient's response to cancer therapy and particularly to Bcl-2 family inhibitor therapy.

The invention comprises assessment in a patient tissue sample of levels of the genes in the biomarker sets, by measurement of these genes at their expressed protein level or translated messenger RNA.

These genomic biomarkers were identified by Applicants through gene expression analysis of human sclc and leukemia/lymphoma cell lines used to test Bcl-2 inhibitors in vitro and in vivo and investigation of their clinical significance. These genomic biomarker combinations are of particular interest for use in companion diagnostic assays to the use of ABT-737 and ABT-263.

Particularly, Applicants identified novel biomarker combinations that discriminate between cell line groups, sclc (See TABLE 1) and leukemia/lymphoma (See TABLE 2) showing sensitivity and resistance to ABT-737.

TABLE 1
SCLC ABT-737 Biomarker Signature Set

| Affymetrix ID | Gene Name | Genbank | Description |
|---|---|---|---|
| 201590_x_at | ANXA2 | NM 004039 | annexin A2 |
| 210427_x_at | ANXA2 | BC001388 | annexin A2 |
| 213503_x_at | ANXA2 | BE908217 | annexin A2 |
| 204693_at | CDC42EP1 | NM 007061 | CDC42 effector protein (Rho GTPase binding) 1 |
| 201605_x_at | CNN2 | NM 004368 | calponin 2 |
| 202894_at | EPHB4 | NM 004444 | EPH receptor B4 |
| 203989_x_at | F2R | NM 001992 | coagulation factor II (thrombin) receptor |
| 210220_at | FZD2 | L37882 | frizzled homolog 2 (Drosophila) |
| 202382_s_at | GNPDA1 | NM 005471 | glucosamine-6-phosphate deaminase 1 |
| 215489_x_at | HOMER3 | AI871287 | homer homolog 3 (Drosophila) |
| 210605_s_at | MFGE8 | BC003610 | milk fat globule-EGF factor 8 protein |
| 204880_at | MGMT | NM 002412 | O-6-methylguanine-DNA methyltransferase |
| 203434_s_at | MME | NM 007287 | membrane metallo-endopeptidase (neutral endopeptidase, |
| | | | enkephalinase, CALLA, CD10) |
| 202443_x_at | NOTCH2 | AA291203 | Notch homolog 2 (Drosophila) |
| 210756_s_at | NOTCH2 | AF308601 | Notch homolog 2 (Drosophila) /// Notch homolog 2 |
| | | | (Drosophila) |
| 212377_s_at | NOTCH2 | AU158495 | Notch homolog 2 (Drosophila) |
| 214722_at | NOTCH2NL | AW516297 | Notch homolog 2 (Drosophila) N-terminal like |
| 205503_at | PTPN14 | NM 005401 | protein tyrosine phosphatase, non-receptor type 14 |
| 212262_at | QKI | AA149639 | quaking homolog, KH domain RNA binding (mouse) |
| 205228_at | RBMS2 | NM 002898 | RNA binding motif, single stranded interacting protein 2 |
| 221016_s_at | TCF7L1 | NM 031283 | transcription factor 7-like 1 (T-cell specific, HMG-box) |
| 212761_at | TCF7L2 | AI949687 | transcription factor 7-like 2 (T-cell specific, HMG-box) |
| 212762_s_at | TCF7L2 | AI375916 | transcription factor 7-like 2 (T-cell specific, HMG-box) |
| 216035_x_at | TCF7L2 | AV721430 | transcription factor 7-like 2 (T-cell specific, HMG-box) |
| 216037_x_at | TCF7L2 | AA664011 | transcription factor 7-like 2 (T-cell specific, HMG-box) |
| 216511_s_at | TCF7L2 | AJ270770 | transcription factor 7-like 2 (T-cell specific, HMG-box) |
| 200931_s_at | VCL | NM 014000 | vinculin |
| 201426_s_at | VIM | AI922599 | vimentin |
| 202133_at | WWTR1 | BF674349 | WW domain containing transcription regulator 1 |
| 211962_s_at | ZFP36L1 | BG250310 | zinc finger protein 36, C3H type-like 1 |

TABLE 2
Leukemia/lymphoma ABT-737 Biomark Signature Set

| Affymetrix ID | Gene Name | Genbank | Description |
|---|---|---|---|
| 201118_at | PGD | NM 002631 | phosphogluconate dehydrogenase /// |
| | | | phosphogluconate dehydrogenase |
| 202779_s_at | UBE2S | NM 014501 | ubiquitin-conjugating enzyme E2S |
| 202950_at | CRYZ | NM 001889 | crystallin, zeta (quinone reductase) |
| 203040_s_at | HMBS | NM 000190 | hydroxymethylbilane synthase |
| 203810_at | DNAJB4 | BG252490 | DnaJ (Hsp40) homolog, subfamily B, member 4 |
| 203911_at | RAP1GA1 | NM 002885 | RAP1, GTPase activating protein 1 |
| 203925_at | GCLM | NM 002061 | glutamate-cysteine ligase, modifier subunit |
| 203946_s_at | ARG2 | NM 001172 | arginase, type II |
| 204624_at | ATP7B | NM 000053 | ATPase, Cu++ transporting, beta polypeptide |
| | | | (Wilson disease) |
| 205164_at | GCAT | NM 014291 | glycine C-acetyltransferase (2-amino- |
| | | | 3-ketobutyrate coenzyme A ligase) |
| 205262_at | KCNH2 | NM 000238 | potassium voltage-gated channel, |
| | | | subfamily H (eag-related), member 2 |
| 205486_at | TESK2 | NM 007170 | testis-specific kinase 2 |
| 206283_s_at | TAL1 | NM 003189 | T-cell acute lymphocytic leukemia 1 |
| 206729_at | TNFRSF8 | NM 001243 | tumor necrosis factor receptor superfamily, member 8 |
| 207522_s_at | ATP2A3 | NM 005173 | ATPase, Ca++ transporting, ubiquitous |
| 208398_s_at | TBPL1 | NM 004865 | TBP-like 1 |
| 209368_at | EPHX2 | AF233336 | epoxide hydrolase 2, cytoplasmic |
| 210036_s_at | KCNH2 | AB044806 | potassium voltage-gated channel, |
| | | | subfamily H (eag-related), member 2 |
| 211673_s_at | MOCS1 | AF034374 | molybdenum cofactor synthesis 1 /// |
| | | | molybdenum cofactor synthesis 1 |
| 212475_at | KIAA0241 | AI797458 | KIAA0241 protein |
| 213036_x_at | ATP2A3 | Y15724 | ATPase, Ca++ transporting, ubiquitous |
| 214696_at | MGC14376 | AF070569 | hypothetical protein MGC14376 |
| 215150_at | YOD1 | AF090896 | YOD1 OTU deubiquinating enzyme |
| | | | 1 homolog (yeast) |
| 215535_s_at | AGPAT1 | AF007145 | 1-acylglycerol-3-phosphate O-acyltransferase |
| | | | 1 (lysophosphatidic acid acyltransferase, alpha) |
| 216317_x_at | RHCE | X63095 | Rhesus blood group, CcEe antigens |
| 218157_x_at | CDC42SE1 | NM 020239 | CDC42 small effector 1 |
| 218617_at | TRIT1 | NM 017646 | tRNA isopentenyltransferase 1 |
| 218647_s_at | YRDC | BE464161 | ischemia/reperfusion inducible protein |
| 218739_at | ABHD5 | NM 016006 | abhydrolase domain containing 5 |
| 219103_at | DDEFL1 | NM 017707 | development and differentiation enhancing factor-like 1 |
| 219578_s_at | CPEB1 | AF329403 | cytoplasmic polyadenylation element binding protein 1 |
| 219611_s_at | CCDC21 | NM 022778 | coiled-coil domain containing 21 |
| 219786_at | MTL5 | NM 004923 | metallothionein-like 5, testis-specific (tesmin) |
| 220150_s_at | C6orf60 | NM 024581 | chromosome 6 open reading frame 60 |
| 220399_at | FLJ22639 | NM 024796 | hypothetical protein FLJ22639 |
| 220807_at | HBQ1 | NM 005331 | hemoglobin, theta 1 /// hemoglobin, theta 1 |
| 221693_s_at | MRPS18A | AB049952 | mitochondrial ribosomal protein S18A /// |
| | | | mitochondrial ribosomal protein S18A |
| 32836_at | AGPAT1 | U56417 | 1-acylglycerol-3-phosphate O-acyltransferase |
| | | | 1 (lysophosphatidic acid acyltransferase, alpha) |
| 217862_at | PIAS1 | N24868 | protein inhibitor of activated STAT, 1 |
| 216221_s_at | PUM2 | D87078 | pumilio homolog 2 (Drosophila) |

Applicants further identified biomarker combinations that show sensitivity and resistance to ABT-263 and further discriminating between cell lines, sclc (See TABLES 3 and 4) and leukemia/lymphoma (See TABLES 5 and 6).

TABLE 3
SCLC ABT-263 BIOMARKER SIGNATURE SET

| Affymetrix ID | Gene Name | Genbank | Description |
|---|---|---|---|
| 210605_s_at | MFGE8 | BC003610 | milk fat globule-EGF |
| | | | factor 8 protein |
| 202443_x_at | NOTCH2 | NM 024408 | Notch homolog 2 |
| | | | (Drosophila) |
| 203435_s_at | MME | NM 007287 | membrane metallo- |
| | | | endopeptidase (neutral |
| | | | endopeptidase, |
| | | | enkephalinase, |
| | | | CALLA, CD10) |
| 210220_at | FZD2 | L37882 | frizzled homolog 2 |
| | | | (Drosophila) |

TABLE 4

| Affymetrix ID | Gene Name | Genbank | Description |
|---|---|---|---|
| 202499_s_at | SLC2A3 | NM 006931 | solute carrier family |
| | | | 2 (facilitated glucose |
| | | | transporter), member 3 |
| 221016_s_at | TCF7L1 | NM 031283 | transcription factor |
| | | | 7-like 1 (T-cell |
| | | | specific, HMG-box) |
| 217730_at | TMBIM1 | NM 022152 | transmembrane BAX |
| | | | inhibitor motif |
| | | | containing 1 |
| 218865_at | MOSC1 | NM 022746 | MOCO sulphurase |
| | | | C-terminal domain |
| | | | containing 1 |

TABLE 5
Leukemia/lymphoma ABT-263 Biomarker Signature Set

| Affymetrix ID | Gene Name | Genbank | Description |
|---|---|---|---|
| 201828_x_at | CXX1 | NM 003928 | CAAX box 1 |
| 205691_at | SYNGR3 | NM 004209 | synaptogyrin 3 |
| 208796_s_at | CCNG1 | BC000196 | cyclin G1 |
| 214696_at | MGC14376 | AF070569 | hypothetical |
| | | | protein MGC14376 |
| 220051_at | PRSS21 | NM 006799 | protease, serine, |
| | | | 21 (testisin) |

TABLE 6

| Affymetrix ID | Gene Name | Genbank | Description |
|---|---|---|---|
| 210775_x_at | CASP9 | AB015653 | caspase 9, apoptosis-related cysteine peptidase |
| 211560_s_at | ALAS2 | AF130113 | aminolevulinate, delta-, synthase 2 (sideroblastic/hypochromic anemia) |
| 217650_x_at | ST3GAL2 | AI088162 | ST3 beta-galactoside alpha-2,3-sialyltransferase 2 |
| 217955_at | BCL2L13 | NM 015367 | BCL2-like 13 (apoptosis facilitator) |
| 204517_at | PPIC | BE962749 | peptidylprolyl isomerase C (cyclophilin C) |
| 201559_s_at | CLIC4 | AF109196 | chloride intracellular channel 4 |
| 208398_s_at | TBPL1 | NM 004865 | TBP-like 1 |
| 209116_x_at | HBB | M25079 | hemoglobin, beta /// hemoglobin, beta |
| 207180_s_at | HTATIP2 | NM 006410 | HIV-1 Tat interactive protein 2, 30 kDa |

III. Assays

The inventive assays include assays both to select patients eligible to receive Bcl-2 family inhibitor therapy and assays to monitor patient response. Assays for response prediction are run before therapy selection and patients with elevated levels are eligible to receive Bcl-2 family inhibitor therapy. For monitoring patient response, the assay is run at the initiation of therapy to establish baseline levels of the biomarker in the tissue sample. The same tissue is then sampled and assayed and the levels of the biomarker compared to the baseline. Where the levels remain the same or decrease, the therapy is likely being effective and can be continued. Where significant increase over baseline level occurs, the patient may not be responding.

The assays of the present invention can be performed by protein assay methods and by nucleic acid assay methods. Any type of either protein or nucleic acid assays can be used. Protein assay methods useful in the invention are well known in the art and comprise (i) immunoassay methods involving binding of a labeled antibody or protein to the expressed protein or fragment of genes in the biomarker set, (ii) mass spectrometry methods to determine expressed protein or fragments of these biomarkers, and (iii) proteomic based or “protein chip” assays. Useful immunoassay methods include both solution phase assays conducted using any format known in the art, such as, but not limited to, an ELISA format, a sandwich format, a competitive inhibition format (including both forward or reverse competitive inhibition assays) or a fluorescence polarization format, and solid phase assays such as immunohistochemistry (referred to as “IHC”).

IHC methods are particularly preferred assays. IHC is a method of detecting the presence of specific proteins in cells or tissues and consists of the following steps: 1) a slide is prepared with the tissue to be interrogated; 2) a primary antibody is applied to the slide and binds to specific antigen; 2) the resulting antibody-antigen complex is bound by a secondary, enzyme-conjugated, antibody; 3) in the presence of substrate and chromogen, the enzyme forms a colored deposit (a “stain”) at the sites of antibody-antigen binding; and 4) the slide is examined under a microscope to identify the presence of and extent of the stain.

Nucleic acid assay methods useful in the invention are also well known in the art and comprise (i) in situ hybridization assays to intact tissue or cellular samples to detect mRNA levels, (ii) microarray hybridization assays to detect mRNA levels, (iii) RT-PCR assays or other amplification assays to detect mRNA levels. Assays using synthetic analogs of nucleic acids, such as peptide nucleic acids, in any of these formats can also be used.

The assay of the present invention also provide for detection of the genomic biomarkers by hybridization assays using detectably labeled nucleic acid-based probes, such as deoxyribonucleic acid (DNA) probes or protein nucleic acid (PNA) probes, or unlabeled primers which are designed/selected to hybridize to the specific designed gene target. The unlabeled primers are used in amplification assays, such as by polymerase chain reaction (PCR), in which after primer binding, a polymerase amplifies the target nucleic acid sequence for subsequent detection. The detection probes used in PCR or other amplification assays are preferably fluorescent, and still more preferably, detection probes useful in “real-time PCR”. Fluorescent labels are also preferred for use in situ hybridization but other detectable labels commonly used in hybridization techniques, e.g., enzymatic, chromogenic and isotopic labels, can also be used. Useful probe labeling techniques are described in Molecular Cytogenetics: Protocols and Applications, Y.-S. Fan, Ed., Chap. 2, “Labeling Fluorescence In Situ Hybridization Probes for Genomic Targets”, L. Morrison et.al., p. 21-40, Humana Press, © 2002, incorporated herein by reference.

A further embodiment the gene expression levels of the biomarker combinations set forth in Tables 1, 2, 3, 4, 5, or 6 can be evaluated using nucleic acid based arrays such as for example cDNA or oligonucleotide arrays, or protein arrays.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectric, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides prepared from one sample, such as peripheral blood, tumor or suspected tumor tissues, or cell isolated such as circulating epithelial cells separated or identified in a blood sample, at a specific time during the course of an anti-cancer treatment, are hybridized to a nucleic acid array. Signals detected after the formation of hybridization complexes indicate that polynucleotide levels in the sample. In one embodiment, the fluorophores Cy3 and Cy5 (Amersham Pharmacia Biotech, Piscataway N.J.) are used as the labeling moieties for the differential hybridization format.

Signals gathered from a nucleic acid array can be analyzed using commercially available software, such as those provided by Affymetric or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained n each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

IV. Sample Processing and Assay Performance

The tissue sample to be assayed by the inventive methods can comprise any type, including a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, and this extract can then be assayed. A microdissection of the tissue sample to obtain a cellular sample enriched with suspected tumor cells can also be used. The preferred tissue samples for use herein are peripheral blood, tumor tissue or suspected tumor tissue, including fine needle aspirates, fresh frozen tissue and paraffin embedded tissue, and bone marrow.

The tissue sample can be processed by any desirable method for performing in situ hybridization or other nucleic acid assays. For the preferred in situ hybridization assays, aparaffin embedded tumor tissue sample or bone marrow sample is fixed on a glass microscope slide and deparaffinized with a solvent, typically xylene. Useful protocols for tissue deparaffinization and in situ hybridization are available from Abbott Molecular Inc. (Des Plaines, Ill.). Any suitable instrumentation or automation can be used in the performance of the inventive assays. PCR based assays can be performed on the m2000 instrument system (Abbott Molecular, Des Plaines, Ill. Automated imaging can be employed for the preferred fluorescence in situ hybridization assays.

In one embodiment, the sample comprises a peripheral blood sample from a patient which is processed to produce an extract of circulating tumor cells having increased expression of the biomarker genes. The circulating tumor cells can be separated by immunomagnetic separation technology such as that available from Immunicon (Huntingdon Valley, Pa.). The number of circulating tumor cells showing altered expression of biomarker genes is then compared to the baseline level of circulating tumor cells having altered expression of biomarker genes determined preferably at the start of therapy.

Test samples can comprise any number of cells that is sufficient for a clinical diagnosis, and typically contain at least about 100 cells.

V. Assay Kits

In another aspect, the invention comprises immunoassay kits for the detection of which kits comprise a labeled antibody or labeled protein specific for binding to genes in the biomarkers set. These kits may also include an antibody capture reagent or antibody indicator reagent useful to carry out a sandwich immunoassay. Preferred kits of the invention comprise containers containing, respectively, at least one antibody capable of binding specifically to at least one of the biomarkers in the set, and a control gene. Any suitable control composition for the particular biomarker assay can be included in the kits of the invention. The control compositions generally comprise the biomarker to be assayed for along with any desirable additives. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding the genes set forth in the biomarker combinations of the present invention. Such kits generally comprise at least one oligonucleotide probe or primer, and preferably oligonucleotide sets corresponding to the biomarker combination groups set out in Tables 1, 2, 3, 4, 5 or 6 as described above that hybridizes to a polynucleotide encoding a protein. Such oligonucleotides may be used, for example, within a PCR or hybridization assay. Additional components that may be present within such kits include a second oligonucleotide, or a set of oligonucleotides corresponding to the biomarker combinations set out in Tables 1, 2, 3, 4, 5 or 6, and/or a diagnostic reagent or container to facilitate the detection of a polynucleotide encoding a tumor protein.

VI. Databases

In yet a further aspect the invention includes relational databases containing sequence information, for instance for one or more of the genes of Tables 1, 2, 3, 4, 5 or 6, as well as gene expression information in various lung cancer and leukemia/lymphoma tissue samples. Databases may also contain information associated with a given sequence or tissue sample such as descriptive information about the gene associated with the sequence information, descriptive information concerning the clinical status of the tissue sample, or information concerning the patient from which the sample was derived. The database may be designed to include different parts, for instance a sequence database and a gene expression database. The databases of the invention may be stored on any available computer-readable medium. Methods for the configuration and construction of such databases are widely available, for instance, see Akerblom et al., (U.S. Pat. No. 5,953,727), which is specifically incorporated herein by reference in its entirety.

The databases of the invention may be linked to an outside or external database. In a preferred embodiment, as described in Tables 1, 2, 3, 4, 5 or 6 the external database is GenBank and the associated databases maintained by the National Center for Biotechnology Information or NCBI (http://www.ncbi.nlm.nih.gov/Entrez/). Other external databases that may be used in the invention include those provided by Chemical Abstracts Service (http://stnweb.cas.org/) or Incyte Genomics (http://www.incyte.com/sequence/index.shtml).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northern blots (E-Northerns) to allow the user to determine the cell type or tissue in which a given gene is expressed and to allow determination of the abundance or expression level of a given gene in a particular tissue or cell. The E-northern analysis can be used as a tool to discover tissue specific candidate therapeutic targets that are not over-expressed in tissues such as the liver, kidney, or heart. These tissue types often lead to detrimental side effects once drugs are developed and a first-pass screen to eliminate these targets early in the target discovery and validation process would be beneficial.

The databases of the invention may also be used to present information identifying the expression level in a tissue or cell of a combination of genes set out in Tables 1, 2, 3, 4, 5 or 6, comprising the step of comparing the expression level of the biomarker combinations set out in Tables 1, 2, 3, 4, 5 or 6 in the tissue to the level of expression of the gene in the database. Such methods may be used to predict the physiological state of a given tissue by comparing the level of expression of the gene combinations set out in Tables 1, 2, 3, 4, 5 or 6 from a sample to the expression levels found in tissue from normal tissue, tissue from tumors or both. Such methods may also be used in the drug or agent screening assays as described herein.

Without further description, it is believed that one of ordinary skill in the art can, using the preceding description and the following illustrative examples, make and utilize the compounds of the present invention and practice the claimed methods. The preceding working examples therefore, are illustrative only and should not be construed as limiting in any way the scope of the invention.

VI. Experimental

### EXAMPLE 1

A genome-wide view of gene expression patterns using microarrays.

### Cell Culture

The following SCLC cell lines were obtained from ATCC (Manassis, Va.): NCI-H889, NCI-H1963, NCI-H1417, NCI-H146, NCI-H187, DMS53, NCI-H510, NCI-H209, NCI-H211, NCI-H345, NCI-H524, NCI-H69, DMS79, SHP77, NCI-H1688, NCI-H446, NCI-H740, NCI-H1048, NCI-H82, NCI-H196, SW1271, H69AR, NCI-H526, NCI-H865, NCI-H748, NCI-H711, and DMS114. All cells were cultured in the ATCC recommended media at 37° C. in a humidified atmosphere containing 5% CO₂. The following leukemia and lymphoma cell lines were obtained from ATCC (Manassis, Va.): MV-4-11, RS4;11, Loucy, KG-1A, DOHH2, Rs11380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, Jurkat I 9.2, Jurkat, MEG-01, U-937, K-562, and Raji.

### Microarray Analysis of Gene Expression

Total RNA was isolated by using the Trizol reagent (Invitrogen,) and purified on RNeasy columns (Qiagen, Valencia, Calif.). Labeled cRNA was prepared according to the microarray manufacturer's protocol and hybridized to human U133A 2.0 arrays (Affymetrix, Santa Clara, Calif.). The U133A 2.0 chips contain 14,500 well-characterized genes, as well as several thousand ESTs. The microarray data files were loaded into the Rosetta Resolver™ software for analysis and the intensity values for all probe sets were normalized using the Resolver's Experimental Definition. The intensity values for the probesets corresponding to genes within the amplified regions were normalized across each gene and compared in heatmaps using the Spotfire™ software.

### Results

The 27 SCLC cell lines were tested for sensitivity to ABT-737 using the procedure described in Oltersdorf, T., “An inhibitor of Bcl-2 family proteins induces regression of solid tumours”, Nature, 435: 677-681, 2005, with a cell line classified as sensitive if its EC50<5 μM and as resistant if its EC50>5 μM. The sensitive cell line group consisted of NCI-H889, NCI-H1963, NCI-H1417, NCI-H146, DMS 53, NCI-H187, NCI-H510, NCI-H209, NCI-H345, NCI-H526, NCI-H211, NCI-H865, NCI-H524, NCI-H748, DMS 79, NCI-H69, NCI-H711, SHP 77, NCI-H1688, and and the resistant cell line group was comprised of NCI-H446, NCI-H740, NCI-H1048, NCI-H82, NCI-H196, SW1271, DMS 114, and NCI-H69AR.

The 22 SCLC cell lines were tested for sensitivity to ABT-263 using the procedure described in Oltersdorf, T., “An inhibitor of Bcl-2 family proteins induces regression of solid tumours”, Nature, 435: 677-681, 2005, with a cell line classified as sensitive if its EC50<5 μM and as resistant if its EC50>5 μM. The sensitive cell line group consisted of NCI-H146, NCI-H889, NCI-H1963, NCI-H187, NCI-H1417, NCI-H211, NCI-H69, NCI-H209, NCI-H510, DMS 53, DMS 79, NCI-H345, NCI-H1048, SHP 77, NCI-H446 and the resistant cell line group was comprised of NCI-H1688, NCI-H740, NCI-H82, NCI-H69AR, SW1271, DMS 114 and NCI-H196.

The 25 leukemia/lymphoma cell lines were also tested for sensitivity to ABT-737 using the 5 uM cut-off, and sensitive cell lines were MV-4-11, RS4;11, Loucy, KG-1A, DOHH2, Rs11380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, and Jurkat I 9.2, and the resistant cell lines Jurkat, MEG-01, U-937, K-562, and Raji.

The 25 leukemia/lymphoma cell lines were also tested for sensitivity to ABT-263 using the 5 uM cut-off, and sensitive cell lines were MV-4-11, RS4;11, Loucy, KG-1A, DOHH2, Rsl 1380, CCRF-HSB-2, CCRF-CEM, CEM/C1, Reh, SUP-B15, MOLT-4, SUDHL4, HL-60, RPMI 8226, A3, Daudi, WSU-NHL, Pfeiffer, and Jurkat I 9.2, and the resistant cell lines Jurkat, MEG-01, U-937, K-562, and Raji.

RNA expression patterns from untreated sclc cell lines and leukemia/lymphoma cell lines were determined using Affymetrix HG-U133A v.2.0 microarrays that contain over 22,000 probe sets. In parallel with separate cultures, we determined the sensitivity of each cell line to the compounds. The expression profiles were divided into sensitive and resistant groups, and a series of statistical filters applied to identify which genes were the best at discriminating between the sensitive and resistant cell lines. The first filter was an Analysis of Variance (ANOVA) using Spotfire software. Variable genes (high CV) were next filtered. The remaining genes were analyzed with JMP's discriminant analysis function to identify the genes that best discriminated between sensitive and resistant cell lines. The best discriminant sets were tested using SAS's leave-one-out cross validation function to identify the best signature set of biomarkers. For ABT-263, 2 sets for each cell type (sclc and leukemia/lymphoma cells) were found to perform well.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A method of identifying a patient for eligibility for cancer therapy comprising: (a) providing a tissue sample from a patient; (b) determining expression levels in the tissue sample of the biomarker combinations set out in TABLES 1, 2, 3, 4, 5 or 6; (c) classifying the levels of expression relative to levels in normal tissue of genes in the corresponding biomarker set; and (d) identifying the patient as eligible to receive a cancer therapy where the patient's sample is classified as having a altered levels of genes in the biomarker set.

2. The method of claim 1, wherein the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

3. The method of claim 2, wherein the periperal blood sample is from a patient with a cancer selected from the group consisting of lung carcinoma and leukemia/lymphoma.

4. The method of claim 2, wherein the tissue sample is a paraffin-embedded fixed tissue sample, a fine needle aspirate or a fresh frozen tissue sample.

5. The method of claim 1, wherein the determining step (b) is performed by in situ hybridization.

6. The method of claim 5, wherein the in situ hybridization is performed with a nucleic acid probe that is fluorescently labeled.

7. The method of claim 5, wherein the in situ hybridization is performed with at least two nucleic acid probes.

8. The method of claim 5, wherein the in situ hybridization is performed with a peptide nucleic acid probe.

9. The method of claim 1, wherein the determining step (b) is performed by polymerase chain reaction.

10. The method of claim 1, wherein the determining step (b) is performed by a nucleic acid microarray assay.

11. The method of claim 1, wherein the patient is classified as eligible to receive an anti-sense therapy compound designed to bind to one of Bcl-2, Bcl-w, and Bcl-x1.

12. The method of claim 1, wherein the cancer therapy comprises a Bcl-2 family inhibitor.

13. The method of claims 11 or 12, wherein the patient is classified as eligible to receive N-(4-=(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

14. The method of claim 11 or 12, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

15. The method of claim 1, wherein the cancer therapy comprises a Bcl-2 family inhibitor in combination with chemotherapy.

16. The method of claim 15, wherein the patient is classified as eligible to receive N-(4-(4-((2-(4-chlorophenyl)-5,5-dimethyl-1-cyclohex-1-en-1-yl)methyl)piperazin-1-Yl)benzoyl)-4-(((1R)-3-(morpholin-4-yl)-1-((phenylsulfanyl)methyl)propyl)amino)-3-((trifluoromethyl)sulfonyl)benzenesulfonamide.

17. The method of claim 15, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

18. A method of identifying a patient for eligibility for Bcl-2 family inhibitor therapy comprising: (a) providing a lung cancer tissue sample from a patient; (b) detecting the level of expression in the tissue sample; wherein differential expression of the biomarker combinations set out in TABLES 1 or 2 is indicative of a patient being eligible to receive Bcl-2 family inhibitor therapy.

19. The method of claim 18, wherein the determining step (b) is performed by PCR.

20. The method of claim 18, wherein the determining step (b) is performed by a nucleic acid microarray assay.

21. The method of claim 18, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

22. The method of claim 18, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

23. The method of claim 18, wherein the patient is classified as eligible to receive an anti-sense therapy compound designed to bind to one of Bcl-2, Bcl-w, and Bcl-xl.

24. The method of claim 18, wherein the cancer therapy comprises a Bcl-2 family inhibitor in combination with chemotherapy.

25. The method of claim 24, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

26. The method of claim 24, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

27. A method of identifying a patient for eligibility for Bcl-2 family inhibitor therapy comprising: (a) providing a leukemia/lymphoma tissue sample from a patient; (b) determining expression levels in the tissue sample of the biomarker combinations set out in TABLES 3, 4, 5 or 6; (c) classifying the level relative to levels in normal tissue of genes in the biomarker set; and (d) identifying the patient as eligible to receive Bcl-2 family inhibitor therapy where the patient's sample is classified as having a altered levels of genes in the biomarker set.

28. The method of claim 27, wherein the determining step (b) is performed by PCR.

29. The method of claim 27, wherein the determining step (b) is performed by a nucleic acid microarray assay.

30. The method of claim 27, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

31. The method of claim 27, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

32. The method of claim 27, wherein the patient is classified as eligible to receive an anti-sense therapy compound designed to bind to one of Bcl-2, Bcl-w, and Bcl-xl.

33. The method of claim 27, wherein the cancer therapy comprises a Bcl-2 family inhibitor in combination with chemotherapy.

34. The method of claim 33, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

35. The method of claim 33, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

36. A method for monitoring a patient being treated with Bcl-2 family inhibitor therapy comprising: (a) providing a peripheral blood sample from a patient; (b) measuring expression levels in the peripheral blood sample of the biomarker combinations set out in TABLES 1, 2, 3, 4, 5 or 6; and (c) determining the expression level relative to a patient baseline blood level of the biomarker combinations set out in TABLES 1, 2, 3, 4, 5 or 6.

37. The method of claim 36, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

38. The method of claim 36, wherein the patient is classified as eligible to receive N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide.

39. A computer system comprising: (a) a database containing information identifying the expression level in lung cancer tissue of a set of genes set out in Table 1, 2, 3, 4, 5 or 6; and (b) a user interface to view the information.

40. A computer system of claim 39, wherein the database further comprises sequence information for the genes.

41. A computer system of claim 39, wherein the database further comprises information identifying the expression level for the genes in normal tissue.

42. A computer system of claim 39, wherein the database further comprises information identifying the expression level for the genes in tissue from a lung tumor.

43. A computer system of any of claims 39-42, further comprising records including descriptive information from an external database, which information correlates said genes to records in the external database.

44. A computer system of claim 43, wherein the external database is GenBank.

45. A method of using a computer system of any one of claims 39-42 to present information identifying the expression level in a tissue or cell of the biomarker combinations set out in Tables 1, 2, 3, 4, 5 or 6, comprising: (a) comparing the expression level of the biomarker combinations set out in Tables 1, 2, 3, 4, 5, or 6 in the tissue or cell to the level of expression of the gene in the database.
